(19) United States
(12) Patent Application Publication
(10) Pub. No.: US 2023/0270664 A1
(43) Pub. Date: Aug. 31, 2023

(54) DELIVERY DEVICE

(71) Applicant: MAX-PLANCK-GESELLSCHAFT ZUR FÖRDERUNG DER WISSENSCHAFTEN E.V., München (DE)

(72) Inventors: Tian Qiu, Stuttgart (DE); Peer Fischer, Freiburg (DE)

(21) Appl. No.: 18/020,339

(22) PCT Filed: Aug. 12, 2021
(86) PCT No.: PCT/EP2021/072510

§ 371 Date: Feb. 8, 2023

(30) Foreign Application Priority Data
Aug. 13, 2020 (WO) PCT/EP2020/072728

(51) Int. Cl. A61K 9/00; A61K 9/50

(52) U.S. Cl. CPC A61K 9/0009; A61K 9/5057; A61K 9/5089; A61K 9/5094

(57) ABSTRACT

The invention relates to a delivery device formed by an aggregation of a plurality of individual particles in a host fluid, wherein one or more individual particles of the plurality of individual particles has a density of less than the host fluid, preferably less than water, and a bonding property which permits the initially separate individual particles to aggregate in said host fluid, i.e. to be connected one to another in said host fluid, to form the aggregation. The invention further relates to a method for producing a plurality of individual particles and to a method of forming a delivery device from a plurality of particles in a host fluid at an aggregation site.

The present invention relates to a delivery device formed by an aggregation of a plurality of individual particles in a host fluid. Furthermore, the invention relates to a method for producing a plurality of individual particles and to a method of forming a delivery device from a plurality of particles in a host fluid.

Passive delivery devices have been widely used in medicine. For example, capsule endoscopes are used to take images of the intestine; drug delivery capsules are used to deliver drugs in a sustained manner. The common drawback of passive devices is that the location of the device or the particle cannot be controlled precisely in the patient's body, which limits the accuracy of diagnostics and the efficacy of treatments. Similarly, liposomes are used for drug delivery, but they are passively distributed by the bloodstream and they cannot enter certain tissues.

Methods exist to actively manipulate small particles in fluids. A spatial gradient of an external physical field is often applied to generate a force to manipulate small (micron to sub-millimeter) particles, which is the case, for example, with optical tweezers, optoelectric tweezers, acoustic tweezers, magnetic tweezers and fluidic tweezers. However, the common limitation of these methods is that the large field gradient can only be realized over short distances and at a rather short distance to the field generator. Such a high gradient is difficult to realize over larger distances as would be necessary, for example, for applications in medicine. Another difficulty is that some fields, like light waves or microwaves, do not easily penetrate biological tissues as they are absorbed. Yet another difficulty is that the power-levels that can be applied are generally restricted due to safety reasons, which limits the forces that can be exerted on small particles.

Finally, it is possible to exert a force on magnetic particles that can be pulled by a magnetic field. However, in the case of static or low frequency magnetic fields, the setups to generate large magnetic field gradients are bulky and the gradients that can be achieved are generally weak. Thus, there is a lack of suitable techniques that can exert a suitably strong force over a distance that is large enough as it is required for certain applications, such as for example biomedical applications that involve the manipulation of small particles and their transport to certain regions in the human body. It is difficult or impractical to maintain suitably high spatial gradients with a physical field that can penetrate deep enough into tissue and that can be established over the required longer distances so that the gradients reach deep enough into the body.

It is therefore an object of the invention to provide a delivery device and a method, which permit a directed transport of the device in a fluid and where a suitable force can be utilized to enable said directed transport.

This object is solved by the subject matter of the independent claims.

In particular, a delivery device formed by an aggregation of a plurality of individual particles in a host fluid is provided, wherein one or more individual particles of the plurality of individual particles has a density of less than the host fluid, preferably less than water, and a bonding property which permits the initially separate individual particles to aggregate in said host fluid, i.e. to be connected one to another in said host fluid, to form the aggregation. The individual particles have a size in at least one dimension selected in the range of 0.1 μm to 1 mm and the device has a size in at least one dimension selected in the range of 1 μm to 10 mm. Hence, in other words, the delivery device according to the invention is formed of a plurality of separate individual particles, i.e. two or more particles, which each can comprise a density less than water. This can allow the delivery particle to float in water, preferably in said host fluid which may have a similar density. Furthermore, the particles comprise a bonding property, which allows the particles to aggregate, i. e. to connect to one another by physical or chemical interactions that are stronger than the thermal forces, which usually keep the particles from connecting for longer periods of time. Because of said bonding property the aggregate, i. e. the delivery device, persists and has a size that is larger than its constituent particles.

As already mentioned above, the individual particles, for example, can have a size in at least one dimension selected in the range of 0.1 μm to 1 mm. In particular they can have a size in the range of 50 μm to 0.8 mm and especially in the range of 100 to 500 μm. The aggregated delivery device, on the other hand, can have a size in at least one dimension selected in the range of 1 μm to 10 mm, in particular in the range of 100 μm to 5 mm, especially in the range of 200 μm to 2 mm.

The bigger size of the delivery device compared to the size of the individual particles results in clear physical changes. For example, the delivery device can be separated from the individual particles by filtration, size-exclusion chromatography and/or gel electrophoresis. The delivery device can also show a different contrast when imaged. For instance, it will scatter light more strongly. If the particles, for example, possess a magnetic property, then the aggregated delivery device will provide a stronger imaging response in magnetic imaging. Furthermore, it can be possible to navigate the delivery device actively through a host body, for example, by applying a physical field. This way, the aggregated delivery device can be actively moved to a specific site.

In this connection it should further be noted that the particles are not necessarily made from a single material, but can be made from a composition of materials, which in combination with one another have the desired properties, i.e. size and/or density and/or porosity and/or magnetic property. For example, the individual particles can be formed by a composition comprising a mixture of magnetic material and an elastomer or the like to form the individual particles, in particular porous particles, optionally encapsulated particles, described herein.

Such a delivery device can be used in fluid environments and the constituents thereof can be transported in the host fluid to an aggregation site by buoyancy where the individual particles can aggregate to form an aggregation either through the application of an external force or an inherent property of the particles. An assembly of the device at an aggregation site then permits the device to be transported from the aggregation site to a target site, if different from the aggregation site, i.e. the aggregation site can either be the target site or a position from which the device is moved to the target site.

If the aggregation site cannot be easily reached due to size constrictions, constituents of the delivery device can be transported individually to the aggregation site where the device is then formed.

According to a first embodiment of the invention the delivery device is a device carrying a cargo that can be deployed at a target site. Thus, it can be possible that the delivery device is configured to transport a cargo, such as a drug, an imaging device, different kinds of tools, imaging contrasts, aids for repairing or dissolving leaks or blockages, respectively, and/or a combination of the above to a target site, where said cargo can be deployed.

It can therefore be possible that said target site is a part of the body, such as a part of the brain, where a certain drug or the like should be delivered to. It can also be possible that the target site is a part of a channel, reservoir, tank or the like, which comprises some kind of clog, which has to be removed, or a leak, which should be sealed. In this case the delivery device can transport, for example, suitable tools to the target said, i.e. the clog or the leak, with which said problem can be solved. Hence, the delivery device can generally transport a variety of different kinds of tools and/or materials.

According to another embodiment the bonding property comprises a magnetic property, which brings about the aggregation of the individual particles. That is, the particles can, for example, be ferromagnetic such that they attract each other once they are in close proximity, for example at an aggregation site, which can be the place where the individual particles aggregate to the delivery device. Thus, there is no need for an active input by a user in order to aggregate the particles to the delivery device.

In yet another embodiment of the invention the bonding property comprises a magnetic property which, on the application of a magnetic field, brings about the aggregation of the individual particles. That is, according to this embodiment, the individual particles aggregate to the delivery device when a magnetic field is applied to the particles. This embodiment has proven to be advantageous if one would like to prevent the individual particles from aggregating spontaneously once they are in close proximity to each other. If the particles comprise the magnetic property, which only brings about the aggregation when a magnetic field is applied, the aggregation process can be controlled actively by a user. Hence, the user can actively decide when and where the particles should aggregate.

In this connection it can also be possible that the magnetic property is actuated in the presence of at least one of a homogenous magnetic field and a non-homogenous magnetic field. Depending on the precise application of the delivery device and/or the materials of which the individual particles are composed of and/or in what kind of “host body” the device should be applied, the type of magnetic field, i.e. whether it is homogeneous or inhomogeneous, can be chosen accordingly. It is also imaginable that both types of magnetic fields are applied for different purposes. For example, it could be possible that, for example, a homogeneous magnetic field is applied in order to aggregate the particles to the delivery device and, in a second step, an inhomogeneous magnetic field is applied to navigate the aggregated device actively through the host fluid to the specific spot, e.g. the target site.

The magnetic field can comprise a field strength in the range of 0.1 mT to 20 T, preferably in the range of 0.1 mT to 10 T. In particular, a field gradient of the applied magnetic field can be in the range of 0.01 T/m to 1000 T/m, preferably in the range of 0.1 T/m 100 T/m.

According to an embodiment of the invention the individual particle is shaped spherical, cylindrical or streamlined or a combination of the foregoing or randomly shaped. Such shapes have proven to be advantageous when used in a fluid.

According to another embodiment the cargo is selected from the group of drugs, genetic materials, contrast agents, viruses, bacteria, cells, polymeric materials, metals or metallic compounds, sensors, cameras, biopsy tools, radioactive materials, reactive chemicals, dyes and colorants, fluorophores, biological materials, needles or a combination of the foregoing and/or a combination of both agents and/or pharmaceutically active compounds and/or biological materials, such as enzymes or genetic materials, blood anticoagulants or blood clotting drugs, such as Heparin or aprotinin, tranexamic acid (TXA), epsilon-aminocaproic acid and aminomethylbenzoic acid, or materials and/or agents configured to seal a leaks or dissolve blockages in pipelines. Hence, the delivery device can be suitable for transportation in different application areas.

According to another embodiment the individual particles are coated with an anti-adhesion layer. The layer prevents the adhesion of the particles to the solid boundary of the host fluids, especially to soft biological tissues. The coating is preferably homogeneous around the external of individual particles. The thickness of the coating is usually less than 100 μm, preferably less than 10 μm, in particular less than 1 μm. The coating can comprise solid, liquid or gas materials or a combination of aforementioned materials. Examples for such materials can be silicone oil, lubricant oil, water, metal, perfluorocarbon, silane, PEG (Polyethylenglycol), PTFE (Polytetrafluorethylene), proteins, lipids, gas, air, Argon, SF6.

It is also possible that the host fluid is the fluid of the urological system, the gastrointestinal system, the nervous system, the blood circulation system, the immune system, the reproductive system, the ophthalmological system or the extracellular system, microfluidics, pipeline systems, fluidic capillaries or fluidic nozzles. (The particles can also comprise a biocompatible and/or biodegradable material, a low-density material, such as oil, gas, polymer, protein-containing materials, vesicles, gas-filled protein nanostructures, aerogels, fibrous materials, carbohydrate-containing materials, multi-materials, highly porous materials and/or or imaging contrast agents such as gas, iodine, barium, gold and/or silver nanoparticles, gadolinium, hyperpolarized gases, vesicles and/or gas-filled protein nanostructures. Especially particles comprising biocompatible and/or biodegradable materials have the advantage that one does not have to worry about the delivery device once it completed its designated task. When introducing the particles, and thus the delivery device, in a host body it can be possible that the delivery device will simply be decomposed and eventually excreted by the body. Particles, which comprise a material with magnetic properties, on the other hand, may also be navigated outside its application area by applying a corresponding physical field, which addresses the delivery device.

In this connection it is noted that low-density materials refer to a category of materials that comprise a density less than the host fluid, preferably less than one tenth of the density of the host fluid. For example, if the host fluid is a water-based solution that has a density range of 1000-1050 kg/m³, then the preferred low-density material comprises a density less than 1000 kg/m³, in particular less than 900 kg/m³, especially less than 500 kg/m³ and more specifically less than 100 kg/m³. Such materials can be, for example, polystyrene (˜75 kg/m³), air (˜1.2 kg/m³) or aerogel (˜1.0 kg/m³).

According to still another embodiment the particles comprise an inherent dipole moment or form a dipole moment on the application of an external field such as for example the magnetic field as described above. Particles comprising or forming a dipole moment can be addressed rather easily by applying a homogeneous and/or inhomogeneous magnetic field. This can for example help to aggregate the particles at a specific spot or also to navigate the aggregated delivery device through the host fluid.

According to a different embodiment of the invention the bonding property comprises a chemical bonding property which, on the application of an external physical field, i.e. infrared light or acoustic field, such as ultrasound, causes the activation of the chemical bonding property to bring about the aggregation of the individual particles. For some particle materials and/or applications it can be necessary when the particles can be addressed with a physical field which causes the activation of a chemical property in order to let the particles aggregate to a delivery device. In some application areas chemical bonds may comprise advantages compared with physical bonds, i.e. the particles can be addressed more easily or the like.

Additionally or alternatively it can be an embodiment of the invention that the chemical bonding property which, on the insertion of the plurality of individual particles into an aggregation environment, i.e. the host fluid, causes the activation of the chemical bonding property to bring about the aggregation of the individual particles. Hence, according to this embodiment the plurality of particles only have to be inserted into the host fluid in order to trigger the aggregation of the particles. That is, in this embodiment the user does not necessarily have to intervene actively to let the particles aggregate. Said aggregation can simply be triggered by the host fluid itself.

A second aspect of the invention relates to a method for producing a plurality of individual particles, wherein the particles are configured to aggregate to a delivery device, preferably to the delivery device according to the invention, wherein the method comprises the steps of mixing a buoyant agent into a first fluid to generate a foaming fluid mixture, mixing the mixture in a second immiscible fluid to generate droplets of a controlled size, and solidifying said droplets. The buoyant agent can be composed of at least one of bubbles, vesicles, gas-filled protein nanostructures, aerogels, colloids, magnetic materials, materials including organic, inorganic and biological materials. The buoyant agent can contribute to the fact that the produced particles are supposed to comprise a density, which is lower than water so that the particles are able to float in water or another fluid, which comprises a similar density than water, e.g. the host fluid. Hence, the buoyant agent can facilitate the motion of the particles in the host fluid. Furthermore, it can help with the formation of aggregates of said particles or with the release of the cargo or maybe even with the efficacy of the application of the delivery device.

The formed droplets can comprise a size in at least one dimension in the range of 0.1 μm to 1 mm. In particular they can have a size in the range of 50 μm to 0.8 mm and especially in the range of 100 to 500 μm.

The foaming fluid mixture comprises at least two phases, i.e. a low-density phase and a fluid phase. The mixture is generated with either a randomly foaming process; or a controlled low-density material encapsulation process, e.g. by forming a gas-containing water droplet using microfluidic droplet generation process. The first fluid and the second fluid are immiscible. Since drugs can be contained in the first fluid, it is often chosen as a compatible fluid for said drug. For example, a water-soluble drug needs a water-based first fluid. Therefore, the second fluid would then be oil-based. Another example can be that the drug is oil soluble such that the first fluid would then be oil-based and the second fluid water-based. The selection process can be a process to choose the desired particles based on one or many of their physical or chemical properties such as their average density, surface chemistry, adhesion force to a particular surface and/or their dimensions.

According to a first embodiment it can also be possible that the method further comprises the step of removing said second fluid to generate the particles out of the solidified droplets. The second fluid can, for example, be removed by a washing process with solvents, e.g. ethanol, acetone, isopropanol. The solvent can then be dried either at room temperature, in a heated oven or in a freeze drying machine.

According to a second embodiment the method further comprises the step of filtering the particles with a selection process. Said particles can, for example, be filtered by size, density, shape or optical properties. One example could be to filter the particles through a filter paper to select a specific size range of particles. Another option could be to mix the particles with a fluid and only choose the particles, which float on said fluid after a given time period, thus filtering the particles by their density. It can also be imaginable to filter the particles by an optical signal, which could be generated in the particle or to filter the particles by centrifugation or to select the particles using an ultrasound or magnetic field such that only particles with specific desired acoustic or magnetic properties are selected, respectively. Hence, the particles can be selected by a plurality of different methods depending on the application.

A third aspect of the invention relates to a method of forming a delivery device from a plurality of particles in a host fluid at an aggregation site, wherein one or more individual particles of the plurality of individual particles has a density of less than water and wherein a size of the each particle in at least one dimension is selected in the range of 0.1 μm to 1 mm, in particular in the range of 10 μm to 0.8 mm, especially in the range of 50 to 500 μm, the method comprising the following steps injecting a particle fluid with a low concentration of the plurality of particles into the host fluid of a fluid containing host; collecting said plurality of particles at said aggregation site following a buoyant passage of said plurality of particles through the host fluid to said aggregation site, with the buoyant passage optionally taking place in a direction opposite to a flow direction of the host fluid; aggregating the plurality of particles at the aggregation site to form the delivery device, wherein a size of the delivery device in at least one dimension is selected in the range of 1 μm to 10 mm, in particular in the range of 100 μm to 5 mm, especially in the range of 200 μm to 2 mm; and navigating the delivery device through the host fluid to the target site.

Thus, it can be seen that when the particles are injected in the host fluid they can float on said fluid and thus, rise up against the direction of gravity. They even experience a buoyant force if the host fluid comprises a flow direction which is exactly opposite to the direction of the buoyant force. Therefore, one can take advantage of the fact that the particles can follow a buoyant passage in order to flow to the aggregation site. Hence, a user does not have to actively intervene in order to bring the particles to the aggregation site, which to date is anyway very difficult because of the small size of the particles.

A possible particle fluid can be air or an inert gas, which comprises a low solubility in the host fluid (e.g. water). Examples for such a fluid are Argon or SF6. The expression “low concentration” refers to a concentration of particles where they do not interact under thermal energy at room temperature. In particular, the concentration should not be higher than 105 particles per millilitre, preferably not even higher than 104 particles per millilitre.

In this connection it is noted also that if the individual particle is too small, the surface force, e.g. fluidic drag and surface interaction, may be stronger than the body force, e.g. gravity and buoyancy, such that the particles will not float. Furthermore, if the individual particle is too small, the buoyancy agent, e.g. gas, may dissolve in the fluid. Hence, the particles should, at least in one direction, be bigger than 1 μm. An advantage of the small size of the individual particles is that they can be injected in places where the bigger devices would not have enough space and would thus clog said place. The particles are small enough that they can follow their buoyant passage until they reach a place big enough for the particles to aggregate to the delivery device.

Currently, delivery device sizes of 200 to 300 μm are required if the device is supposed to be imaged with state of the art imaging devices such as MRI, X-ray or the like. However, in general the device size can also be chosen to be smaller. Thus, when the known imaging techniques become better, also smaller devices can be imaged. Also, if no imaging technique is required for the application of the delivery device, it can also already be smaller than 200 μm. Hence, it can be seen that the size of the delivery device can be chosen with respect to the application of said device.

Regarding the navigation of the delivery device it is noted that in step of navigating the delivery device, it is either possible that the device floats in the flow direction of the host fluid to the target site or that it is navigated by applying an external physical field such as a magnetic field, with which the delivery device is moved in any given direction in the host fluid, even against the flow direction and/or gravity and/or buoyancy, to the target site.

Said navigation via buoyancy can also be supported by change of orientation of the host body, i.e. by moving the host body. This can be helpful if, for example, the particles have to flow through a channel, which comprises curves and/edges. By changing the orientation of the whole host body with respect to the gravitational field, the relative direction of the buoyancy force can be influenced. Thereby it is possible to direct the movement of said particles or said delivery device in both speed and direction.

According to a first embodiment the method comprises the further step of deploying a cargo carried by said particles at the target site, wherein during said step of deploying said cargo the particles optionally develop a density higher than water.

The cargo carried by the particles can be of different nature, such as drugs, genetic materials, contrast agents, viruses, bacteria, cells, polymeric materials, sensors, cameras, biopsy tools, radioactive materials, reactive chemicals, biological materials, liposomes, nanoparticles, needles or a combination of the foregoing and/or a combination of both agents and/or pharmaceutically active compounds and/or biological materials, such as enzymes or genetic materials, blood anticoagulants or blood clotting drugs, such as heparin or aprotinin, tranexamic acid (TXA), epsilon-aminocaproic acid and aminomethylbenzoic acid or materials configured to seal a leak or dissolve a blockage in pipelines. Hence, the delivery device can function as a transportation device for different kinds of applications.

By developing a density higher than water the particles, and thus the device, can experience sedimenting force and move in the direction of gravity, which may even be against flow direction of fluid. It is also possible that after deploying the cargo carried by said particles, the attractive force, which holds the delivery device together, gets smaller such that the device falls apart into separate individual particles again. This can be helpful since single particles can be decomposed more easily than a bigger device.

According to another embodiment said step of aggregating the plurality of particles and/or said step of navigating the delivery device and or said step of deploying a cargo is controlled by applying an external field, force or a torque, such as a light field, a magnetic field, an acoustic field, an electric field, an electromagnetic field, a chemical field or a combination of the foregoing, by changing an average density, the shape, the orientation, e.g. by moving a host body of the host fluid, the adhesion force to a solid boundary or a combination of the foregoing. Hence, the aggregation as well as the navigation can be triggered and/or supported by applying at least one of the above mentioned external fields.

It is possible that the plurality of particles further comprise an imaging contrast agent such that the delivery device can be detected at the aggregation and/or the target site by imaging methods, such as ultrasound, X-ray, CT, MRI, PET, magnetic particle imaging, fluorescence imaging. It can also be possible that the particles comprise magnetic properties such that the particles themselves form the imaging contrast agent.

The invention will now be described in further detail by way of example only with reference to the accompanying drawings. In the drawings there are shown:

FIG. 1: an exemplary schematic of the steps of delivering particles in a fluidic environment;

FIG. 2: an exemplary schematic of the steps of delivering particles in the central nervous system;

FIG. 3: an exemplary schematic of the particle;

FIG. 4: an exemplary schematic of the steps of fixing the position of the cluster;

FIG. 5: an exemplary schematic of the steps of changing the angle between the channel in the host body;

FIG. 6: an embodiment of the delivered particle in a spherical shape;

FIG. 7: an embodiment of the delivered particle in a streamlined shape;

FIG. 8: an embodiment of the delivered particle in a cylindrical tube shape;

FIG. 9: an embodiment of the delivered particle with a tail;

FIG. 10: an embodiment of the delivered particle with multiple buoyant agents;

FIG. 11: embodiment of the delivered particle with a porous matrix;

FIG. 12: a microscopic image of delivered particles;

FIG. 13: the production workflow of the said particles according to the invention;

FIG. 14: an exemplary swelling process of the said particles; and

FIG. 15: an exemplary filtration process of the produced particles.

### BRIEF DESCRIPTION OF THE FIGURES

FIG. 1 illustrates an exemplary schematic of the steps of delivering particles in a fluidic environment, i.e. a host fluid, wherein dispersed particles 100 are driven by buoyancy due to their reduced density with respect to the fluid, which is typically composed of water, and move up the long and thin channel 400 to a chamber, i.e. an aggregation site AS. The particles 100 then aggregate at the aggregation site AS into a cluster 200, i.e. the delivery device 200, which is actuated by a second field to a target site. The delivery device 200 then undergoes a cargo deployment state 300 at the target site TS.

FIG. 2 illustrates a possible application of the invention. It illustrates an exemplary schematic of the steps of delivering particles 100 in a central nervous system to a brain stem, wherein dispersed particles 100 are driven by buoyancy in the cerebral spinal fluid and move up the subarachnoid space 500 to the aggregation site AS. The particles then aggregate into a cluster 200 at the subarachnoid cisterns and the cluster 200 is actuated by a second field to move to the target site TS. The drug, i.e. the cargo, carried by the particles 100 is released in the deployment state 300 at the target site TS at the top of the brain.

FIG. 3 illustrates an exemplary schematic of a particle 100, on which a force or a torque from a second field is exerted in order to let the particle 100 move laterally or rotate to avoid obstacles 510 along its moving path during the buoyancy-driven step.

FIG. 4 illustrates an exemplary schematic of the steps of fixing the position of the cluster 200 at the deployment state 300 at the target site TS in a brain, by an external field generator 800. Said external field generator 800 could, for example, be a magnetic or an ultrasonic transducer.

FIG. 5 illustrates an exemplary schematic of the steps of changing the angle between a channel 400 in a human body, in which the individual particles 100 move in, and the direction of gravity to control the velocity of the particles 100. In the example shown, the angle is changed by adjusting the angle of the bed 700, on which the person lies on. By changing said angle, also the angle between the buoyancy direction and the direction of gravity is changed such that the velocity of the particles 100 can be controlled.

FIG. 6 shows an embodiment of a delivered particle 100 in a spherical shape, which consists of a buoyancy agent 120, magnetic particles 130 and therapeutic agents 140 embedded in a biodegradable gel 110. A magnetic gradient force is used in this embodiment to move the device laterally.

FIG. 7 shows one embodiment of a delivered particle 100 in a streamlined shape, i.e. an oval shape or rugby ball shaped configuration. In FIG. 7(a) one can see how the particle 100 moves against the direction of gravity when no magnetic field is applied, whereas FIG. 7(b) shows how a lateral velocity component is realized due to an asymmetric fluid drag when a magnetic torque is applied to tilt the particle 100.

FIG. 8 shows another embodiment of a delivered particle 100 in a cylindrical tube shape. In this embodiment the buoyant agent 120 gradually dissolves in the fluid through the interfaces 150 such that the average density of the particle increases overtime, which facilitates the recovery of the particle.

FIG. 9 shows an embodiment of a particle 100 with a tail. A magnetic torque is applied to bend the tail 160, which results in a lateral velocity component due to the asymmetric fluid drag.

FIG. 10 shows an embodiment of the particle 100 with multiple buoyant agents 120. The materials can be gas, oil or low-density polymers.

FIG. 11 shows an embodiment of a particle 100 with a porous matrix 110. In this embodiment the low-density material 120 is filled in the porous matrix 110.

FIG. 12 shows a microscopic image of a delivered particle 100 with multiple buoyant agents.

FIG. 13 shows an embodiment of the production workflow of the said particles 100. FIG. 13(a) shows how a water-based gelatin solution is mixed with magnetic powders and cargos. In FIG. 13(b) a foam is generated with the water phase solution. Then, in FIG. 13(c) another immiscible phase, e.g. an oil phase, is prepared such that then (see FIG. 13(d)) the two phases are thoroughly mixed to generate an emulsion. In FIG. 13(e) the water phase is solidified to form solid particles. After forming the particles one can see in FIG. 13(f) that a filtration process can be used to select the particles of a certain kind. Afterwards the oil phase can be removed and the selected particles are dried (FIG. 13(g)).

FIG. 14 shows an embodiment of a swelling process of the said particles. FIG. 14(a) shows a microscopic image of said particles 100 in the dried form while FIG. 14(b) shows the size distribution of the dried particles 100. In FIG. 14(c) one can see a microscopic image of said particles 100 in an aqueous solution in the swelled form while FIG. 14(d) shows the corresponding size distribution of the swelled particles 100.

FIG. 15 shows an embodiment of the filtration process of the produced particles 100. FIG. 15(a) shows that the particles float in the opposite direction of the gravity and accumulate at the water-air interface. The particles that reach the interface within a given time period are selected. FIG. 15(b) shows a microscopic image of the selected particles with a narrower size distribution and a higher porosity.

In the following different embodiments of how the particles 100 and thus also the delivery device 200 according to the invention can be moved and navigated inside the host fluid and how they can be produced are described.

### DETAILED DESCRIPTION OF THE PREFERRED EMBODIMENTS

### Embodiment 1: Particles Delivery Controlled by a Magnetic Field

In one embodiment, the particles comprise of a buoyant agent 120, e.g. a gas bubble, an oil droplet, a low-density polymer, vesicles, gas-filled protein nanostructures; a magnetic agent, e.g. micro- or nano-particles of Fe3O4, Fe, Co, Ni, FePt, NdFeB, permalloy; and a cargo. The particles have an average density lower than the host fluid that they move in and the dispersed particles 100 move against the gravity direction through a thin channel 400. During the buoyancy-driven moving step, there may be obstacles 510 of different kinds in said channel 400. There may also be friction and/or adhesion between the particle 100 and the channel wall. An external magnetic force or torque can be applied to the particles 100 to actuate the motion of the particles 100 to avoid the obstacles 510 or the adhesion and keep moving along the channel 400.

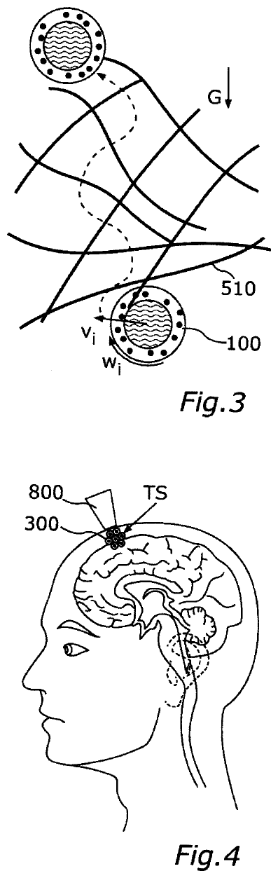

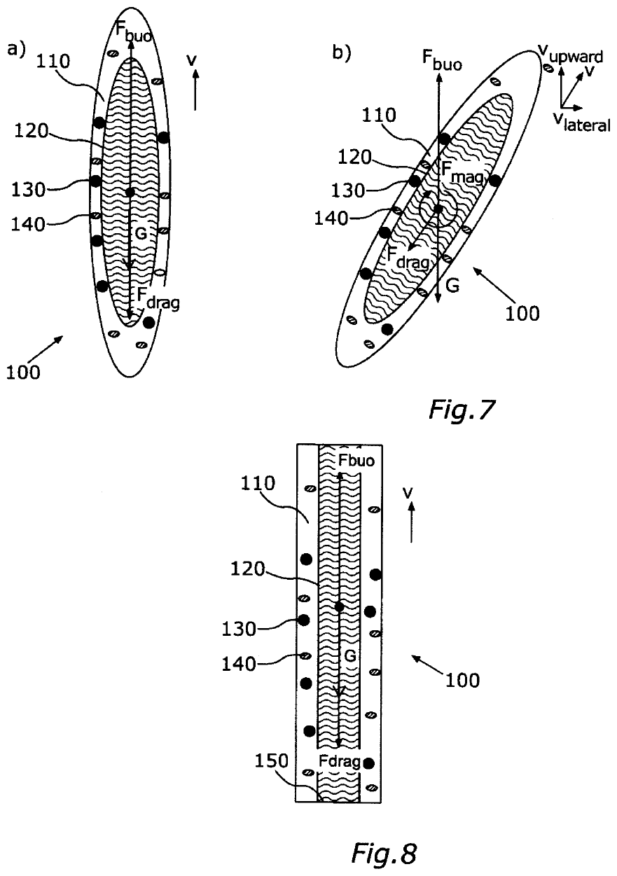

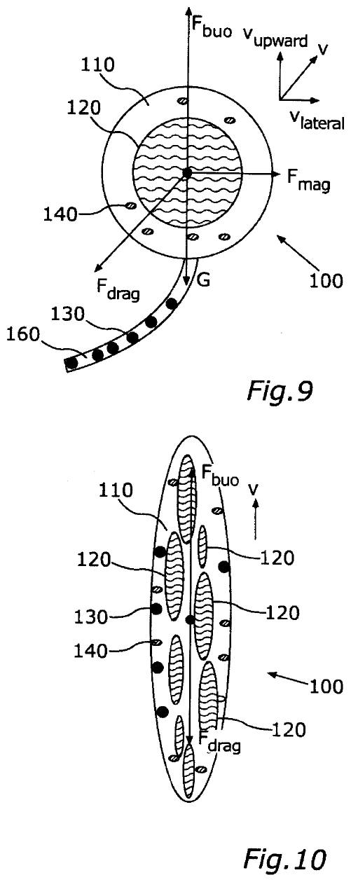

In one embodiment, a magnetic gradient can be applied to the particles 100 to exert a lateral force that moves the particles 100 sideways and to avoid the obstacles 510, as illustrated in FIG. 3 or FIG. 6. In another embodiment, a magnetic field can be applied to the particles 100 to change the orientation of the particles 100 or a part of the particle. For example, in FIG. 7, the orientation of the particle changes and the particle 100 exhibits an asymmetric fluidic drag force, so that it has a lateral moving velocity. In another example, in FIG. 9, a flexible part of the particle 100, i.e. a tail, is bent due to the magnetic torque such that the tail exhibits an asymmetric fluidic drag force and the particle 100 a lateral moving velocity. In yet another example, the magnetic torque drives the rotation of the particle 100 or a small particle cluster. The rolling motion can lead to lateral movements when the particle or the cluster 200 is in touch with an obstacle 510 or a wall, in order to move around the obstacles 510. By changing the direction of the rolling motion, the translational movement direction can be controlled so as to avoid the obstacles 510.

In some embodiments, the magnetic field can be generated by a permanent magnet or the combination of several permanent magnets, where the orientation or the position of the magnet can be static or dynamic. In some embodiments, the magnetic field can be generated by an electromagnetic coil and/or a combination of multiple electromagnetic coils or one or multiple superconducting coils. It is a preferred embodiment that the rolling motion caused by a rotating magnetic field and the magnetic field gradient acting on the particles or clusters move them in the same direction. The field strength of the magnetic field is preferred to be lower than 20 T, more preferably lower than 10 T. The field gradient of the magnetic field is preferred to be lower than 1000 T/m, more preferably lower than 100 T/m.

In some embodiments, it is not necessary to image or localize the dispersed particles 100. More specifically, the relative position of the particles 100 to the obstacles 510 can be unknown. A random magnetic field can be applied to the particles 100 to move them stochastically to avoid said obstacles 510 in the channel 400. In some embodiments, the particles 100 can be localized with an imaging method to determine the position and/or the relative position to the obstacle 510, for example by merging several medical imaging modalities. A control system is then applied on the external magnetic field to actively move the particles 100 in the desired direction to avoid the obstacles 510.

When the particles 100 have moved through the narrow channel 400, they exhibit a second step where the particles 100 aggregate to a cluster 200, which is typically larger in size than the individual dispersed particle 100 and typically caused by attractive interactions between the particles 100. The attractive interactions can, for example, be caused by the magnetic moments of the particles 100. The delivery device 200 then exhibits a larger magnetic moment than the individual particle 100. Thus, it is easier to manipulate the delivery device 200 by an external magnetic field or field gradient. Moreover, the delivery device 200 has a higher mass and a larger overall size. Thus, it is easier to detected the delivery device 200 by medical imaging, such as MRI, CT, X-ray, magnetic particle imaging and ultrasound imaging, than the individual particles 100. In addition, the delivery device 200 and the particles 100 can contain contrast agents 120 or physical characteristics that facilitate the observation of the particle 100 or the particle aggregate 200 by an imaging method.

Said aggregate 200 can be moved actively, for example, under the actuation of a magnetic field or a field gradient. A control system on the external magnetic field can be applied to actively move the agglomerate to a desired target site TS, e.g. a tumor location. An alternative is that the said aggregate 200 can move passively, for example, with a physiological flow, e.g. blood flow, cerebral spinal fluid (CSF) flow, lymphatic flow, urinary flow in the body. Furthermore, a magnetic field gradient at a desired aggregation site AS can enrich the particles 100, as illustrated in FIG. 4. The particles 100 can be guided or enriched at multiple locations depending on the needs and application.

Said particle 100 can also exhibit paramagnetism, superparamagnetism or ferromagnetism. The magnetic properties of the particles 100 can also be used to generate heat by the application of an alternating magnetic field, which can be used to trigger the release of the cargo in the cargo deployment step.

In one embodiment, the particles 100 exhibit a magnetic property and aggregate in the presence of a magnetic field. Specifically, a homogeneous magnetic field can be applied by a magnetic field generator based on permanent magnets (a magnetic flux density is ˜100 mT at the center of the workspace and the field is homogeneous within ±10% in the area of 20 mm×20 mm). Microscopic videos show that the particles 100 aggregate in the presence of the magnetic field and due to their buoyancy. When the external magnetic field rotates, e.g. at 100 rpm (round per minute), the particle aggregates 200 also rotate at the same speed. Due to the irregular shape of the aggregates 200, the aggregates 200 roll on a solid surface, i.e. the solid-liquid boundary. The locomotion of the particle aggregates 200 can be used to avoid solid obstacles when moving in a fluidic channel 400. Decreasing the magnetic flux density from 100 mT to 4 mT still results in similar aggregation and rotation of the particles 100 or the particle aggregates 200.

Alternatively, the magnetic field can also be generated by electromagnetic coils, e.g. Helmholtz coils. The magnetic field may be static or alternative, may be homogeneous or inhomogeneous either spatially or temporally.

In yet another embodiment a permanent magnet, or several permanent magnets, or an electromagnetic coil or several electromagnetic coils can be applied to cause an inhomogeneous magnetic field that facilitates the aggregation of said particles 100. The inhomogeneous magnetic field may exert a magnetic gradient force on said particles 100 or aggregates 200 that cause their locomotion. For example, as illustrated in FIG. 4, an aggregate can be formed with the presence of a permanent magnet 800 close to the neck of a patient. After the dispersed particles 100 pass through the spinal canal by buoyancy, the magnet 800 can be moved from the neck to the top of the head close to the target site TS such that the aggregate 200 of the particles is dragged to the target location TS for targeted drug delivery.

In yet another embodiment, the particles 100 or aggregates 200 flow together with the biological flow in the biological lumen and accumulate at the target location TS due to the presence of an external magnetic field gradient. For example, as illustrated in FIG. 4, after dispersed particles pass through the spinal canal by buoyancy, they flow with the CSF to the upper part of the brain and accumulate at the target location TS due to the presence of a permanent magnet 800 at the close location out of the skull.

### Embodiment 2: Particles Delivery Controlled by an Acoustic Field

In some embodiments, the buoyant agents 120 of the particle 100 can also serve as a contrast agent for an acoustic field due to the different acoustic impedances of materials. For example, gas bubbles exhibit a lower acoustic impedance than water or biological tissues and they accumulate at the anti-node of an acoustic field. Such contrast agents can be used to exert strong acoustic manipulation forces on the particles 100 to avoid obstacles or to move them to a desired location AS, TS. Furthermore, they can also be used to enhance an ultrasound imaging contrast to detect or localize, for example, the particles 100 or aggregates 200. The preferred frequency range of the acoustic field is in the range of 20 kHz to 100 MHz, preferably below 20 MHz, which can penetrate biological tissues.

Said aggregates 200 can move actively, for example, under the actuation of an ultrasonic field or an ultrasonic field gradient. A control system on the external acoustic field can be applied to actively move the aggregate 200 to a desired location TS, e.g. a tumor location. An alternative is that said aggregate 200 can move passively, for example with a physiological flow such as a blood flow, CSF flow, lymphatic flow, urinary flow in the body. A local ultrasonic field generated by an ultrasound transducer can enrich the particles 100 at a desired location AS, as illustrated in FIG. 4. The particles 100 can be guided or enriched at multiple locations AS, TS depending on the needs and applications.

In some embodiments, ultrasound energy can also be applied to and absorbed by the particles 100 to generate heat, which can be used to trigger the release of the cargo in the cargo deployment step.

In some embodiments, ultrasound energy can be combined with the magnetic energy to manipulate the particles 100 or release their cargos.

### Embodiment 3: Particles Delivery Controlled by Changing the Angle Between the Channel and the Direction of Gravity

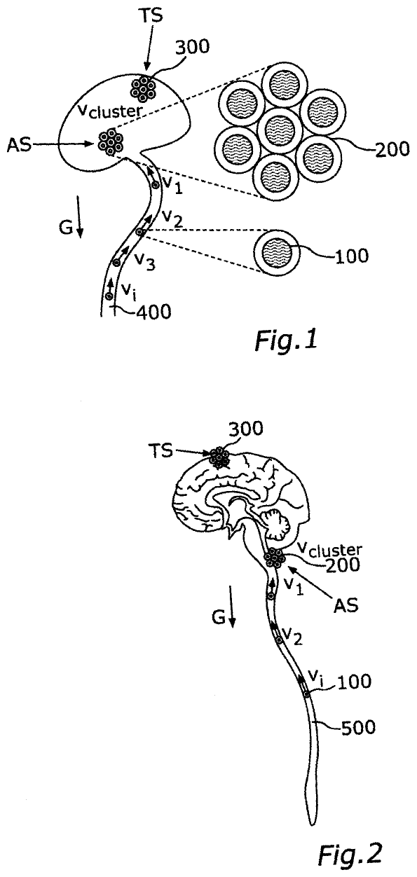

In one embodiment, the velocity of the particles 100 can be controlled by changing the angle between the channel 400 in which they move in and the direction of gravity. When the channel 400 is in a vertical direction that is parallel to the gravity direction, the buoyancy-driven moving velocity reaches its maximum. By changing the angle of the channel 400, the particle's moving direction can be altered relative to the direction of the channel 400. Using this method, it is possible to guide the particles 100 along a branched and/or a complicated shaped channel 400, such as the one shown in FIG. 1, or to avoid some obstacles 510 in the channel 400, such as shown in FIG. 3. It is preferred that changing the direction of the channel 400 is achieved by rotating the channel 400 around a fixed center point with a controlled angular velocity. The preferred rotational angle is not more than 180°, more preferably not more than 90°.

In some embodiments, the preferred orientation for the injection of the particles 100 is in a horizontal orientation, which is perpendicular to the gravity direction. The channel 400 is gradually tilted from the horizontal to the vertical orientation under the control of a motorized stage to start the moving step. The preferred angular velocity is lower than 180°/s, preferably lower than 90°/s, in particular lower than 45°/s. The preferred tilting directions are bidirectional, i.e. the rotation can be in both clockwise and anti-clockwise directions.

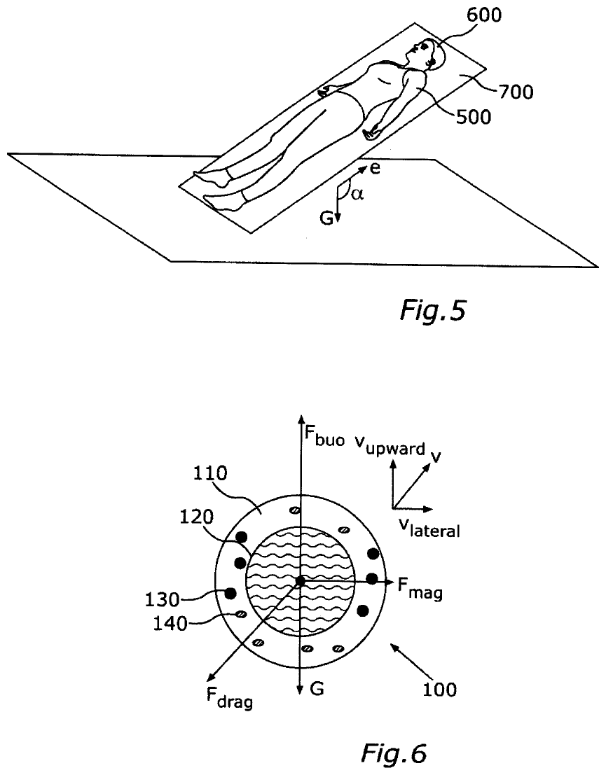

In some embodiments, said particle delivery procedure can take place in the urinary system, in the blood circulation system or in the central nervous system of a host body, as illustrated in FIGS. 2, 4 and 5. The rotation of the host body or a part of the host body is used to guide the movements of the particles 100 in complicated environments or to change the velocity of the particles 100 or clusters 200. In one embodiment, the bed 700, in which the host body lies on, can be tilted relative to the direction of gravity, as illustrated in FIG. 5. The channel 400, which is inside the body of the host body, is thus also tilted, which facilitates the particles' movement towards the target site TS. This procedure can be applied both to upward moving buoyancy-driven particles 100 as well as to downward moving sedimentation-driven particles 100. During the delivery procedure, the rotation of the bed 700 can be dynamically changed to guide the particles 100 to follow a complicate-shaped channel 400 or trajectory, as illustrated in FIGS. 2 and FIG. 4. It is preferred that in the buoyance-driven particles 100 the head of the host body is at a higher position than the rest of the body. The angle of the bed 700 can be controlled by a motorized system and can rotate in both clockwise and anti-clockwise directions. A control algorithm can adapt medical imaging data of the host body, which may also be facilitated with a real-time localization of the said particles 100 or particle aggregates 200.

### Embodiment 4: Particles Delivery in the Urinary System

In one embodiment, the delivery method is applied in the urinary tract. For example, the dispersed particles 100 travel through the long and thin ureter of a patient, which comprises, on average, an inner diameter of 1 mm and a length of around 30 mm. Then, the particles 100 aggregate in the collecting system of the kidney for further manipulation, imaging or cargo deployment.

In one embodiment, the particles 100 are attached to the renal calculi and move the calculi to a safer location to clear an obstruction of the urinary tract to avoid an emergency surgery. An alternative method is to move the calculi into the tool channel of an endoscope, into a gripper or out of the body for clearance.

In another embodiment, the particles aggregate to a larger cluster 200 in the collecting system of the kidney. Then they can release a contrast agent to be localized with a medical imaging such as X-ray or ultrasound imaging. The aggregate 200 is moved actively by a second field, e.g. an acoustic field or a magnetic field, wirelessly to a desired target site TS, e.g. a tumor location, to release drugs or other pharmaceutical agents.

In another embodiment, the particles 100 are injected at a desired location of the blood vascular system, preferably in a peripheral vascular system. Then, the dispersed particles 100 move to another desired location in the vascular system, i. e. the aggregation site AS, where they aggregate to a larger cluster 200 (delivery device 200) to block the blood flow. For example, the device 200 can be used for prostatic artery embolization (PAE), i.e. a minimally invasive treatment that helps to improve the Benign Prostatic Hyperplasia (BPH). The aggregate 200 can then be moved to, or more preferably can be fixed, at the desired location, i. e. the target site TS, by a second field, e.g. an acoustic field or a magnetic field, against the blood flow to induce embolization.

### Embodiment 5. Particles Delivery in the Nervous System

In some embodiments, the delivery device is used inside the nervous system of a patient. The nervous system includes the peripheral nervous system and the central nervous system that consists of the brain and the spinal cord. The particles 100 can carry pharmaceutical agents or medical devices to a desired location AS, TS in the nervous system.

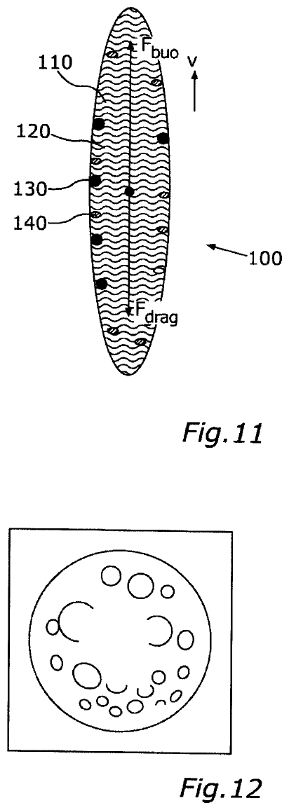

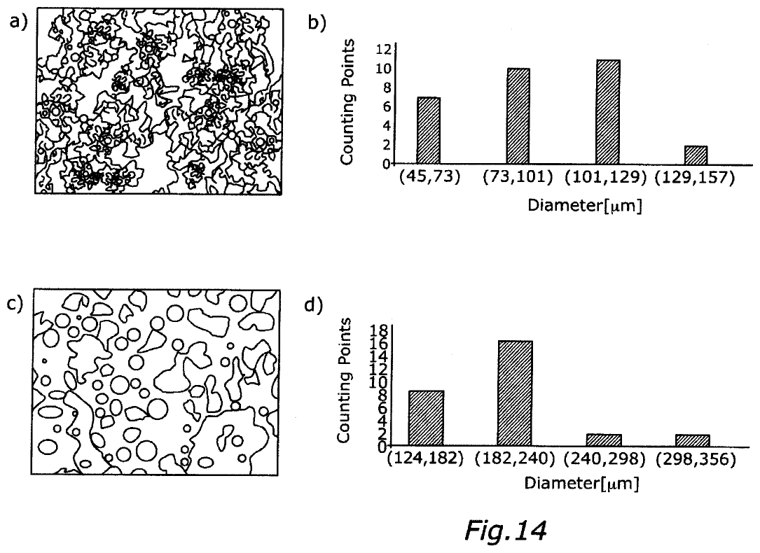

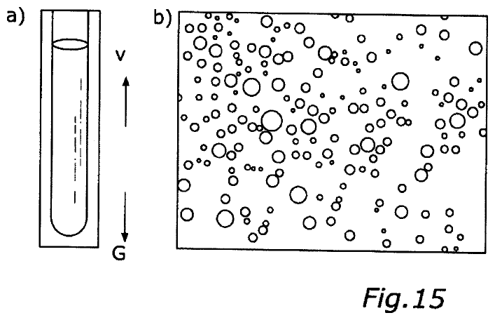

In one embodiment, the particles are made of biocompatible hydrogel material, e.g. alginate, agar, Pluronic, or gelatin hydrogel, and they contain a buoyancy agent and a cargo as illustrated in FIG. 6 to FIG. 11 and shown in the microscopic images in FIG. 12, FIG. 14a, FIG. 14c and FIG. 15b. The particles can be injected into a blood vessel or the central nervous system, preferably with a non-magnetic needle or tube that induces a relatively low shear stress on said particles during the injection process. The preferred injection orientation of the patient is horizontal, i.e. the patient is lying on a bed 700 with the spinal cord approximately in the horizontal plane perpendicular to the direction of gravity. The bed 700 of the patient can be tilted, as illustrated in FIG. 5, and the dispersed particles 100 move up the spine 500, as illustrated in FIG. 2. The particles 100 can avoid obstacles 510, for example, the trabecular, the nerves and the blood vessels in the subarachnoid space of the spine, either passively or actively under the control of a second field, e.g. a magnetic field or an acoustic field.

In one embodiment, the particles 100 are delivered to a desired location AS, TS in the spinal cord such as a desired nerve or a nerve root, where drugs or biological materials are released.

In some embodiments, the particles 100 move through the CSF along the spinal canal to the brain, where the particles 100 aggregate to a cluster 200, for example by the magnetic interactions of the particles 100. The aggregate 200 can be manipulated with a second field, e.g. a magnetic field or an acoustic field, to reach a desired location TS in the brain. In one embodiment, the particles 100 flow with the CSF to the cerebral hemisphere and accumulate at the target site TS, where the pharmaceutical agent can be released. The aggregate 200 preferably stays at the target site TS during said deployment step. It is preferred to use biocompatible and biodegradable materials, e.g. hydrogels, iron or iron oxide, FePt, to fabricate the particles 100. In some embodiments, biological non-degradable or even toxic materials, e.g. nickel, cobalt, can also be used to produce the particles. Then, an additional recovery step for these toxic materials can be applied. For example, the materials can flow out of the brain with the CSF to the lower sections of the spine and be collected by a needle or a magnetic probe. The materials can also be manipulated with a second field, e.g. a magnetic or an acoustic field, to reach a desired location other than the target location TS and facilitate the easy removal process of the non-degradable or toxic materials.

In some embodiments, the particle aggregate 200 exerts high enough force in the second field that it can penetrate soft tissues, e.g. brain tissues, so that the aggregate 200 can move into a deeper target location TS in the biological tissues to release the cargos.

The target diseases in the nervous system, which can be treated with the delivery device 200 and the methods according to the invention include but not limited to the following: diseases caused by faulty genes, such as muscular dystrophy, problems with the development of the nervous system, such as spina bifida, degenerative diseases, such as Parkinson's disease and Alzheimer's disease, diseases of the blood vessels in the brain, such as strokes, injuries to the spinal cord and brain, seizure disorders, such as epilepsy, cancer, such as brain tumors, infections, such as meningitis, and other diseases that can be similarly treated by the methods proposed in this invention.

Said delivery method can be used in the development and testing of drugs and medical devices, for example in the clinical or pre-clinical studies. In some embodiments, it can facilitate the delivery of cargo to the central nervous system in an animal experiment. The application is either to test the efficacy of the drugs or devices, or to induce certain disease to generate a diseased animal model.

### Embodiment 6: In Vitro Applications

In another embodiment, the particles 100 can be used in an in vitro environment, for example, in a microfluidic channel, or a lab-on-a-chip device. The particles are injected at the inlet of the channel 400 and the direction of the channel 400 relative to the gravity direction is changed to control the dispersed particles 100 to move in said channel 400 with a desired velocity. The process facilitates the passing through narrow openings or narrow tubes and it does not require any additional field for actuation. When the particles 100 reach the desired location AS, for example, a larger chamber, the particles 100 can assemble under a second field, for example, a magnetic field or an ultrasonic field, so that the aggregate 200 of particles 100 exhibits larger actuation force and stronger imaging contrast.

Said particles 100 can then be used for the sample preparation in a lab-on-a-chip device. For example, the particles 100 can be chemically functionalized with desired molecules, such as DNA or antibodies to capture desired materials in the biological medium. Due to the low density of the particles 100, they are easily separated from other materials that do not bind to the particles. The delivery device 200 can facilitate further enrichment of the sample or manipulation of the cluster 200 of the particles to a desired location TS for the next step of chemical reactions. The captured cargos can be released or deployed at the desired location TS for a better detection or other analysis. The binding biological materials can be a cell, for example circulating tumor cells (OTC); a molecule, for example, a protein or a DNA; a bacterium or an organism.

Said particles 100 can also be injected in narrow fluidic channels in the industry, for example, in the pipelines of oil or food industry, in an automobile system, in an airplane, in a hydraulic system to detect or repair an abnormality of the fluidic channels. The motion of the particles is driven by density difference of the particles to the fluid and no additional field needs to be applied. When the particles reach the desired location AS, TS or detect the abnormality, the aggregate 200 can be formed or the cargo can be released to repair the problem or to send out a signal to localize the problem.

### Embodiment 7: Aggregation by Chemical Properties

In one embodiment, the aggregation is triggered by a particular chemical environment, e.g. pH change; the presence of certain dissolved ions, biological molecules, including DNA (Deoxyribonucleic acid), RNA (Ribonucleic acid), viruses, and proteins or the presence of certain organisms. For example, a particular kind of antigen, e.g. immunoglobulin, is originated from an infectious disease of the CNS (central nervous system), the coating of the surface of individual particles with specific antibodies can cause the aggregation of the particles at the infectious location in the presence of the antigen in the CSF (cerebral spinal fluid). In another example, specific antibodies, which bond with the coat molecules of certain kind of bacteria, can be coated on the surface of individual particles such that the aggregation of the particles occurs in the presence of the bacteria, or more specifically the particles aggregate around the bacteria. In another embodiment the presence of biological molecules, antigens, RNA, proteins, and/or viruses triggers a chemical reaction on the surface of the particles that facilitates their aggregation and bonding.

In some embodiments, said chemical environment may also trigger the deployment of the cargo, e.g. release of a drug, in the presence of said chemical clues. For example, antibiotics and/or other antimicrobial agents that are included in the matrix of the particles can be released, only when the surface of the particles are chemically triggered and aggregate on the surface of bacteria. In such a way, the effectiveness of the drug is maximized and the delivery of the drug is targeted.

In one embodiment, the particles are made of thermal-responsive gels, e.g. gelatin, agarose gel, poly(N-isopropylacrylamide (PNIPAM), polyvinylalkohol (PVA) gel. In another embodiment, the particles are made of polymer materials that are coated with thermally-activated cross-linkers, e.g. HEMA (hydroxyethyl methacrylate), HEA (2-Hydroxyethyl acrylate), Mba (N,N′-Methylenebisacrylamide), Formaldehyde, Glutaraldehyde, or photo-activated cross-linkers, e.g. 2,2-dimethoxy-2-phenylacetophenone (DMPA). Light or acoustic energy absorbing materials and/or a gas are preferably mixed in the particles. In the presence of light, preferably infrared light that can penetrate biological tissues; and/or in the presence of an acoustic field that can penetrate biological tissues, heat is generated at the particles due to the absorbing of the energy of the physical fields, which triggers the aggregation of individual particles and/or facilitates the bonding of the particles in the formation of the aggregate.

In some embodiments, the chemical bonding property can be implemented in conjunction with magnetic property or acoustic property. For example, the magnetic force or the acoustic radiation force causes the accumulation of individual particles. The chemical bonds are triggered at the surface of particles to bring about the aggregation of the particles. The bonding can also be triggered by transfer of energy from a time-changing magnetic field to the magnetic property of the particles, which causes their heating and facilitates their aggregation and/or their bonding after they have been aggregated. For instance, a static or quasi-static magnetic field first induces the aggregation and then a time changing magnetic field (ac magnetic field with a frequency for instance of at least 1 kHz or preferably 100 kHz) heats the particles which releases a chemical or softens or melts a chemical contained within the particles such that different particles can aggregate.

### Embodiment 8: Particle Fabrication by an Emulsion Method

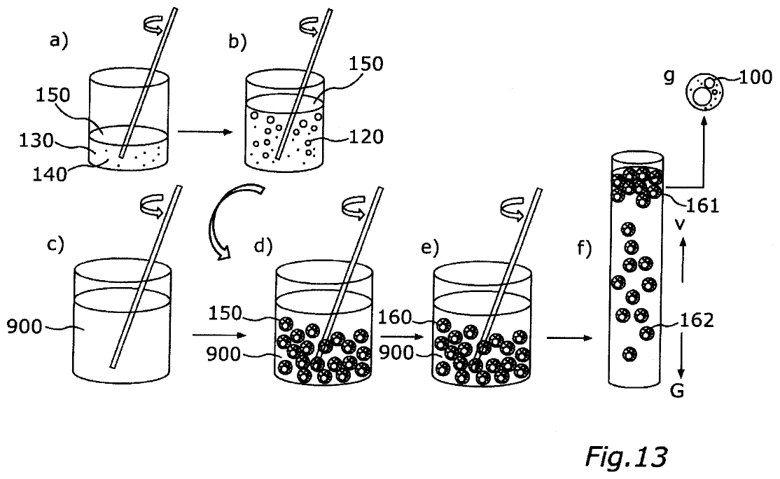

One embodiment of the method of forming said particles using an emulsion method is illustrated in FIG. 13. First, water-based gelatin solution 150 (20% w/v) is prepared at 60° C. 400 rpm and mixed with magnetic powder 130 (3% w/v) and cargo 140, which, for example, can be pharmaceutical agents or drugs (0.01 mg/mL). The solution goes through a foaming process, where a foaming agent, i.e. Na₂CO₃ (10 mg/ml) in acid solution (1 mM), is added to the solution under continuous stirring at 400 rpm. The generated foam contains many gas bubbles 120 with controllable sizes in the range of 0.1 μm to 100 μm diameter in the water phase. The foam is mixed with a preheated silicone oil phase 900 (317667, Sigma-Aldrich, volume ratio 1:100) at 60° C. 400 rpm to generate a water-in-oil emulsion. The aqueous droplets 150 contain gas bubbles 120, magnetic powder 130 and suitable cargo 140 with a controllable size. The solution is stirred and cooled down in an ice bath to room temperature to generate solid hydrogel particles 160. A crosslinking process of the gelatin with glutaric dialdehyde (10%) solution can be used at this step. The emulsion is then washed with solvents, i.e. ethanol, three times to remove the oil phase 900 and the particles 100 are dried in an oven at 60° C. The particles were examined under bright field optical microscopy. The corresponding pictures and size distributions of the particles can be found in FIG. 14.

An additional filtration process can be added to select the particles 100 with desired size, density, shape or optical properties. A common process is to filter the particles through a filter paper to select their size range. In another process, as shown in the FIG. 15a, a column is filled with aqueous solution, e.g. 0.9% NaCl solution, and the particles float to the air-solution interface due to their buoyancy. A given time period, e. g. 60 seconds for a distance of 120 mm, is then used to select the particles of desired density. For example, the particles with a fast floating velocity 161, e. g. a floating velocity larger than 2 mm/s, are selected and the particles, which do not float, or the ones with a slow floating velocity 162 are filtered out. An image showing the process is illustrated in FIG. 15a. The microscopic image in FIG. 15b shows that the particles after the said selection process comprise a narrower size distribution and a higher porosity.

In some embodiments, the particles can be selected due to an optical signal generated in the particle, for example the fluorescence signal of the cargos. In some embodiments, the particles can be selected using a centrifugation process, or more preferably a density matching centrifugation process. In some embodiments, the particles can be selected using an ultrasound field, only particles with desired acoustic properties are selected. In some embodiments, the particles can be selected using a magnetic field, only particles with desired magnetic properties are selected.

### Experimental Results

One particular example of a particle 100 according to the invention is shown in the microscopic picture of FIG. 12. The particle comprises an at least substantially spherical shape with a diameter of approximately 200 μm, after the suspension and swelling in 0.9% NaCl solution. The particle 100 comprises a solid body made of gelatin-based hydrogel 110 and multiple gas (air) bubbles of different diameters in the range of 1-50 μm as buoyant agents 120. Fluorophores (Rhodamine 6G, 252433, Sigma-Aldrich) and magnetic microparticles 130 are also encapsulated in the hydrogel 110, which could not be visualized in the FIG. 12 due to the small size of the microparticles and fluorophores 130.

Under low-frequency (in the range of a static field up to 1 kHz) magnetic field, the particles 100 and or the particle 100 in its host fluid aggregates to a delivery device 200, which can then be pulled by the magnetic field gradient (typically in a gradient range of 1 T/m to 500 T/m) or be rotated by the temporally rotating spatially-homogeneous magnetic field (typically at a field strength range of 1 mT to 1 T), to reach the target site TS. At the target site TS a high-frequency magnetic field (typically with a frequency higher than 1 kHz and a field strength higher than 1 mT) is applied to generate heat on the magnetic microparticles 130 inside the particles 100. When the temperature of the particles 100 or the particle aggregates 200 is higher than a certain temperature threshold, for example the melting point of the gelatin hydrogel (40° C.), the gel matrix 110 melts and the load 140 is released at the target site TS.

In order to produce such a particle 100 as described above, the following method steps according to the invention have been carried out:

First, a buoyant agent 120 is mixed into a first fluid to generate a foaming fluid mixture. A foaming agent, i.e. for example Na₂CO₃ (10 mg/ml), is added to the water-based solution with acid (1 mM) under continuous stirring at 400 rpm. The water-based solution was a gelatin hydrogel solution (G1890, Sigma-Aldrich, 20% w/v), which had been prepared at 60° C. 400 rpm and mixed with magnetic powder 130 (Nickel, GF14196067, Sigma-Aldrich, 3% w/v) and cargo 140 (Rhodamine 6G, 252433, Sigma-Aldrich, 0.01 mg/mL). The foaming process generates multiple gas (CO₂) bubbles in the water-based solution. The gas bubbles size distribution can be controlled via appropriate choice of fluidic viscosity, temperature additive and chemicals concentration, stirring speed, fluidic shear rate, surface tension and so on.

In a next step, the mixture is mixed with a second immiscible fluid to generate droplets of a controlled size. Therefore, the mixture from step 1 has been mixed with a preheated second oil-phase fluid 900 (317667, Sigma-Aldrich, volume ratio 1:100) at 60° C. 400 rpm to generate a water-in-oil emulsion. The aqueous droplets 150 containing the gas (CO₂) bubbles 120, magnetic powder 130 and suitable cargo 140 with a controllable size have then been dispersed in the oil. The particle size can be can be controlled via appropriate choice of fluidic viscosity, temperature, additive and chemicals concentration, stirring speed, fluidic shear rate, surface tension and so on.

Afterwards, the solution has been stirred and cooled down in an ice bath to room temperature to generate solid hydrogel particles 160. A crosslinking process of the gelatin with glutaric dialdehyde (10%) solution has then been added at this step, which typically lasts overnight.

The emulsion generated from step 3 is then washed with solvents, i. e. for example ethanol, for three times to remove the oil phase 900 and in order to let the particles 100 dry in an oven at 60° C. in air. Gas exchange happens at this step, and it generates porous microparticles filled with air.

In order to filter the produced particles 100, a glass container (approximately 120 mm in length) was filled with 0.9% NaCl solution and placed in the direction of gravity (as shown in FIG. 15a). Then, the prepared particles 100 have been injected at the bottom of the container and collected at the top of the solution surface within a time period of, for example 60 s. Thus, particles 100 of an average low density, i.e. a large rising velocity in solution of not smaller than 2 mm/s, could be selected. The selected particle suspension can go through an additional filtering process through filter papers to filter out particles 100 of certain size range, e.g. 100-200 μm in diameter. Finally, the particles are dried again in the oven and stored in a sealed container at 4° C.

1.-20. (canceled)

21. A delivery device formed by an aggregation of a plurality of individual particles in a host fluid, wherein one or more individual particles of the plurality of individual particles has a density of less than the host fluid, and a bonding property which permits the initially separate individual particles to aggregate in said host fluid to form the aggregation, wherein the individual particles have a size in at least one dimension selected in the range of 0.1 μm to 1 mm, and the device has a size in at least one dimension selected in the range of 1 μm to 10 mm.

22. The delivery device according to claim 21, wherein the delivery device is a device carrying a cargo that can be deployed at a target site.

23. The delivery device according to claim 21, wherein the bonding property comprises a magnetic property, which brings about the aggregation of the individual particles.

24. The delivery device according to claim 21, wherein the bonding property comprises a magnetic property which, on the application of a magnetic field, brings about the aggregation of the individual particles.

25. The delivery device according to claim 24, wherein the magnetic property is actuated in the presence of at least one of a homogenous magnetic field and a non-homogenous magnetic field.

26. The delivery device according to claim 24, wherein the magnetic field comprises a field strength in the range of 0.1 mT to 20 T.

27. The delivery device according to claim 21, wherein the individual particle is shaped spherical, cylindrical, streamlined or a combination of the foregoing or randomly shaped.

28. The delivery device according to claim 22, wherein the cargo is selected from the group of drugs, genetic materials, contrast agents, viruses, bacteria, cells, polymeric materials, metals or metallic compounds, sensors, cameras, biopsy tools, radioactive materials, reactive chemicals, dyes and colorants, fluorophores, biological materials, needles or a combination of the foregoing and/or a combination of both agents and/or pharmaceutically active compounds and/or biological materials, such as enzymes or genetic materials or materials configured to seal a leak or dissolve a blockage in pipelines.

29. The delivery device according to claim 21,
wherein the particles are coated with an anti-adhesion layer.

30. The delivery device according to claim 21, wherein the host fluid is the fluid of the urological system, the gastrointestinal system, the peripheral and the central nervous system, the cerebral spinal fluid, the blood circulation system, the immune system, the reproductive system, the ophthalmological system, the extracellular system, microfluidics, pipeline systems, fluidic capillaries or fluidic nozzles.

31. The delivery device according to claim 21, wherein the particles comprise a biocompatible and/or biodegradable material, a low density material, such as oil, gas, polymer, protein-containing materials, vesicles, gas-filled protein nanostructures, aerogels, fibrous materials, carbohydrate-containing materials, multi-materials, highly porous materials, and/or or imaging contrast agents, such as gas, iodine, barium, gold and/or silver nanoparticles, gadolinium, hyperpolarized gases, vesicles and/or gas-filled protein nanostructures.

32. The delivery device according to claim 21, wherein the particles comprise an inherent dipole moment or form a dipole moment on the application of an external field.

33. The delivery device according to claim 21, wherein the bonding property comprises a chemical bonding property which, on the application of an external physical field, i.e. infrared light or acoustic field, such as ultrasound, causes the activation of the chemical bonding property to bring about the aggregation of the individual particles; and/or
wherein the chemical bonding property which, on the insertion of the plurality of individual particles into an aggregation environment, i.e. the fluid, causes the activation of the chemical bonding property to bring about the aggregation of the individual particles.

34. A method for producing a plurality of individual particles, wherein the particles are configured to aggregate to a delivery device, wherein the method comprises the steps of:
mixing a buoyant agent into a first fluid to generate a foaming fluid mixture;
mixing the mixture in a second immiscible fluid to generate droplets of a controlled size; and
solidifying said droplets.

35. The method according to claim 34, wherein the method further comprises the step of:
removing said second fluid to generate the particles out of the solidified droplets.

36. The method according to claim 34, wherein the method further comprises the step of:
filtering the particles with a selection process.

37. A method of forming a delivery device from a plurality of particles in a host fluid at an aggregation site, wherein one or more individual particles of the plurality of individual particles has a density of less than water and wherein a size of the each particle in at least one dimension is selected in the range of 0.1 μm to 1 mm,
the method comprising the following steps:
injecting a particle fluid with a low concentration of the plurality of particles into the host fluid of a fluid containing host;
collecting said plurality of particles at said aggregation site following a buoyant passage of said plurality of particles through the host fluid to said aggregation site, with the buoyant passage optionally taking place in a direction opposite to a flow direction of the host fluid;
aggregating the plurality of particles at the aggregation site to form the delivery device, wherein a size of the delivery device in at least one dimension is selected in the range of 1 μm to 10 mm; and
navigating the delivery device through the host fluid to the target site.

38. The method according to claim 37, wherein the method comprises the further step of
deploying a cargo carried by said particles at the target site, wherein during said step of deploying said cargo the particles optionally develop a density higher than water.

39. The method according to claim 37,
wherein said step of aggregating the plurality of particles and/or said step of navigating the delivery device and or said step of deploying a cargo is controlled by applying an external field, force or a torque, a magnetic field, an acoustic field, an electric field, an electromagnetic field, a chemical field or a combination of the foregoing, by changing an average density, the shape, the orientation, the adhesion force to a solid boundary or a combination of the foregoing.

40. The method according to claim 37,
wherein the plurality of particles further comprise an imaging contrast agent such that the delivery device can be detected at the aggregation and/or the target site by imaging methods.
